(19) United States
(12) Patent Application Publication
(10) Pub. No.: US 2026/0083893 A1
(43) Pub. Date: Mar. 26, 2026

(54) AIR EXTRACTION DEVICE AND NASAL ASPIRATOR

(71) Applicant: Zhongshan Xingfu Industry and Trade Co., Ltd., Zhongshan (CN)

(72) Inventor: Qiang ZHANG, Zhongshan (CN)

(73) Assignee: Zhongshan Xingfu Industry and Trade Co., Ltd., Zhongshan (CN)

(21) Appl. No.: 19/361,228

(22) Filed: Oct. 17, 2025

(30) Foreign Application Priority Data
Sep. 23, 2024 (CN) 202422321577.4

(51) Int. Cl. A61M 1/00

(52) U.S. Cl. CPC A61M 1/684; A61M 2202/0014; A61M 2202/04; A61M 2210/0618

(57) ABSTRACT

An air extraction device and a nasal aspirator. The air extraction device includes a cylinder body and a variable-volume body. The cylinder body defines a cavity and a first air hole, and the first air hole is in communication with the cavity. The variable-volume body is arranged within the cavity. The variable-volume body is configured such that, through a change in a volume of the variable-volume body within the cavity from large to small, an air pressure within the cavity is reduced, for providing a suction force externally through the first air hole.

### CROSS REFERENCE

The present application claims priority of Chinese Patent Application No. 202422321577.4, filed on September 23, 2024, the entire contents of which are hereby incorporated by reference in its entirety.

### TECHNICAL FIELD

The present disclosure relates to an air extraction device and a nasal aspirator.

### BACKGROUND

A nasal aspirator is a device configured to extract nasal mucus, primarily categorized into electric-driven and manual-driven types. The manual-driven nasal aspirators have the advantages of relatively simple structure and lower cost. However, in the related art, a suction structure of an existing manual nasal aspirator is prone to wear. After prolonged use, air tightness will decrease, leading to a reduction in suction force. Additionally, the structures of this nasal aspirator are often designed for single-use connections, making them difficult to clean and maintain regularly.

### SUMMARY OF THE DISCLOSURE

A first aspect of the embodiments of the present disclosure provides an air extraction device, including: a cylinder body; wherein the cylinder body defines a cavity and a first air hole, and the first air hole is in communication with the cavity; and a variable-volume body, arranged within the cavity; wherein the variable-volume body is configured such that, through a change in a volume of the variable-volume body within the cavity from large to small, an air pressure within the cavity is reduced, for providing a suction force externally through the first air hole.

In some embodiments, the cylinder body defines an opening; the variable-volume body includes a pipe body; wherein the pipe body includes a first end and a second end, and the second end of the pipe body is arranged with a bottom wall; wherein the first end of the pipe body is capable of sealing the opening of the cylinder body, and a side wall of the pipe body is capable of expanding and of contracting within the cavity.

In some embodiments, the first end of the pipe body is arranged with a flange that is annular in shape and extends radially outward; an outer diameter of the flange is greater than an inner diameter of the opening of the cylinder body, and the first end of the pipe body is capable of sealing the opening of the cylinder body via the flange.

In some embodiments, the side wall of the pipe body is capable of expanding and of folding along an axial direction of the pipe body.

In some embodiments, the air extraction device further includes a pull rod assembly; the pull rod assembly includes a rod body, and a first end of the rod body is inserted through the first end of the pipe body and connected to the bottom wall of the pipe body; the rod body is configured to drive the pipe body to expand or contract by pushing or pulling the rod body.

In some embodiments, the opening of the cylinder body is arranged with a cover, and the cover is configured to restrict the flange at the opening of the cylinder body.

In some embodiments, the air extraction device further includes a second air hole and a first one-way valve; the second air hole is disposed on the pipe body or the cylinder body; a first end of the second air hole is in communication with the cavity of the cylinder body, and a second end of the second air hole is connected to the first one-way valve; wherein gas within the cavity is capable of being discharged unidirectionally through the first one-way valve.

In some embodiments, the bottom wall of the pipe body defines a second through hole; the second through hole is arranged with a connecting member, and the second air hole is disposed on the connecting member; the rod body is arranged with an air passage, and a side wall of the rod body defines a third air hole; wherein a first end of the air passage is in communication with the second air hole, and a second end of the air passage is in communication with the third air hole.

In some embodiments, an outer wall of the first end of the rod body is arranged with a locking block; the connecting member is arranged with a connecting column, and the connecting column defines an insertion hole; wherein the insertion hole is in communication with the second air hole; a side wall of the insertion hole defines a guide groove along an axial direction of the insertion hole; a side wall of the connecting column near the connecting member defines a lock hole, and the lock hole is opened along a peripheral direction of the connecting column; wherein the guide groove is in communication with the lock hole; the rod body is capable of being inserted into the insertion hole, and the locking block is movable along the guide groove to a set position; in a case where the rod body is relatively rotated, the locking block moves toward an end of the lock hole.

In some embodiments, the air extraction device further includes an elastic member; the elastic member is configured to drive the variable-volume body to expand or contract through a restoring force of the elastic member.

In some embodiments, the first air hole is connected to a second one-way valve, and the cavity of the cylinder body is capable of unidirectionally drawing in air through the second one-way valve.

A second aspect of the embodiments of the present disclosure provides a nasal aspirator, including the aforementioned air extraction device, a liquid storage tank, and an air guide tube; wherein the liquid storage tank includes a chamber; a first end of the liquid storage tank defines a liquid inlet hole, and the liquid inlet hole is in communication with the chamber; a side wall of the liquid storage tank defines a fourth air hole, and the fourth air hole is in communication with the chamber; a first end of the air guide pipe is in communication with the first air hole of the air extraction device, and a second end of the air guide pipe is in communication with the fourth air hole of the liquid storage tank.

In some embodiments, the liquid storage tank further includes a conduit; the conduit is disposed within the chamber, and a first end of the conduit is connected to the liquid inlet hole.

### BRIEF DESCRIPTION OF THE DRAWINGS

The accompanying drawings, which form a part of the present disclosure, provide further understanding of the present disclosure. The illustrative embodiments and descriptions herein are intended to explain the present disclosure and do not constitute an undue limitation.

FIG. 1 is a schematic structural view of a nasal aspirator according to some embodiments of the present disclosure.

FIG. 2 is a schematic structural view of an air extraction device according to some embodiments of the present disclosure.

FIG. 3 is an exploded view of FIG. 2.

FIG. 4 is a cross-sectional view taken along line A-A of an air extraction device shown in FIG. 1, showing the device in a first state.

FIG. 5 is a cross-sectional view taken along line A-A of the air extraction device shown in FIG. 1, showing the device in a second state.

FIG. 6 is a schematic structural view of a variable-volume structure according to some embodiments of the present disclosure.

FIG. 7 is a schematic cross-sectional view of a variable-volume body according to some embodiments of the present disclosure.

FIG. 8 is a cross-sectional view of the variable-volume body with a spring installed, as shown in FIG. 4.

FIG. 9 is a cross-sectional view of the variable-volume body with a spring installed, as shown in FIG. 5.

FIG. 10 is a cross-sectional view taken perpendicular to line A-A of the air extraction device shown in FIG. 1.

FIG. 11 is a schematic structural view of a rod body according to some embodiments of the present disclosure.

FIG. 12 is a schematic structural view of a connecting member according to some embodiments of the present disclosure.

FIG. 13 is a schematic view of the connecting member in FIG. 12 from another perspective.

FIG. 14 is a cross-sectional view taken along line B-B of FIG. 13.

FIG. 15 is an exploded view of a liquid storage tank according to some embodiments of the present disclosure.

FIG. 16 is a cross-sectional view of FIG. 15.

FIG. 17 is a schematic view of another embodiment of the liquid storage tank.

### DETAILED DESCRIPTION

The present disclosure will now be described in detail with reference to the accompanying drawings and embodiments. The examples are provided to explain the present disclosure and not to limit it. In practice, those skilled in the art will recognize that modifications and variations may be made to the present disclosure without departing from its scope or spirit. For example, features shown or described as part of one embodiment may be used in another embodiment to produce a further embodiment. Therefore, it is intended that the present disclosure covers such modifications and variations falling within the scope of the appended claims and their equivalents.

In the description of the present disclosure, terms such as “longitudinal”, “transverse”, “upper”, “lower”, “front”, “rear”, “left”, “right”, “vertical”, “horizontal”, “top”, and “bottom” indicate directions or positional relationships based on the drawings. These terms are used for ease of description and do not require the present disclosure to be constructed or operated in a specific orientation. Thus, they should not be construed as limiting the present disclosure. The terms “connected”, “linked”, and “arranged” are broadly interpreted. For example, connections may be fixed or detachable, direct or indirect via an intermediate component, and may involve wired, wireless, or signal-based links. Those skilled in the art will understand the specific meanings of these terms based on context.

The accompanying drawings illustrate one or more examples of the present disclosure. The detailed description uses numerical and alphabetical references to denote features in the drawings. Similar or analogous references in the drawings and description refer to similar or analogous parts of the present disclosure. Terms such as “first”, “second”, and “third” are used interchangeably to distinguish one component from another and do not indicate position or importance.

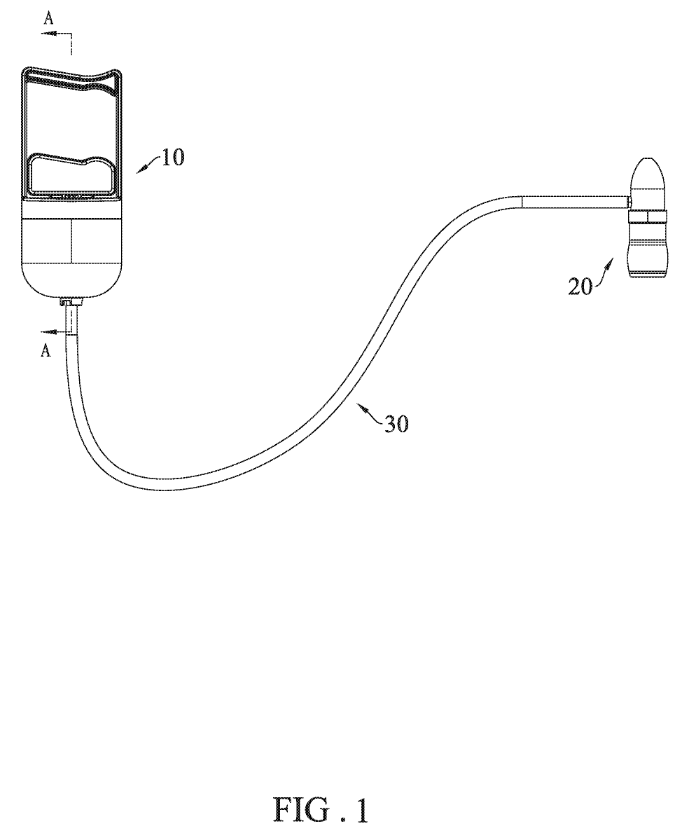

As shown in FIG. 1, some embodiments of the present disclosure provide a nasal aspirator, which includes an air extraction device 10, a liquid storage tank 20, and an air guide pipe 30. The air guide pipe 30 is connected to the air extraction device 10 and the liquid storage tank 20 on both ends.

The present disclosure discloses an air extraction device 10, which includes a variable-volume structure 11 and a cylinder body 12. The cylinder body 12 defines a cavity 1201 with an open end, and the open end is disposed on a first end of the cylinder body 12 to define an opening of the cylinder body 12. A cylinder bottom 1203 is arranged on a second end of the cylinder body 12. The cylinder bottom 1203 defines a first air hole 1202 in communication with the cavity 1201. The variable-volume structure 11 includes a variable-volume body 110 disposed in the cavity 1201. A volume of the variable-volume body 110 is capable of selectively increasing and decreasing within the cavity 1201. A first end of the variable-volume body 110 away from the cylinder bottom 1203 seals the opening of the cylinder body 12. When the volume of the variable-volume body 110 in the cavity 1201 increases, air pressure in the cavity 1201 rises, forcing air out through the first air hole 1202. When the volume of the variable-volume body 110 in the cavity 1201 decreases, the air pressure in the cavity 1201 drops, drawing outside air into the cavity 1201 through the first air hole 1202.

In some embodiments, the variable-volume body 110 includes a pipe body 15 that is length adjustable and defines an inner cavity 1501, and the inner cavity 1501 has an open end on a first end of the pipe body 15. The first end of the pipe body 15 seals the opening of the cylinder body 12, while a second end of the pipe body 15 is arranged with a bottom wall 1505. A side wall 1504 of the pipe body 15 is capable of expanding and contracting along an axial direction of the pipe body 15, allowing the second end of the pipe body 15 to move toward or away from a bottom of the cylinder body 12, thereby achieving volume change within the cylinder body 12.

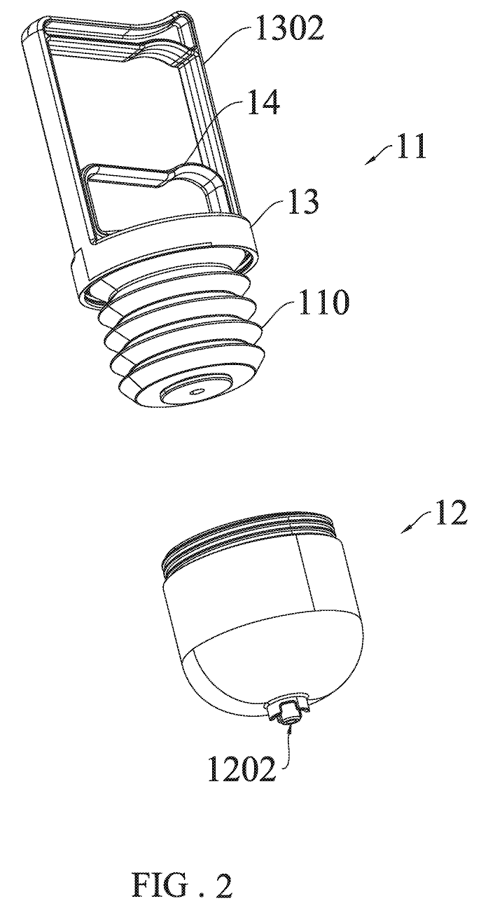

As shown in, FIGS. 2, 3, and 7, in some embodiments, the first end of the pipe body 15 is arranged with a flange 1503 that is annular in shape and extends outward along a radial direction of the pipe body 15. An outer diameter of the flange 1503 is greater than an inner diameter of the opening of the cylinder body 12. In this way, when the second end of the pipe body 15 is inserted into the cylinder body 12, the flange 1503 abuts and tightly connects with the opening of the cylinder body 12, thereby sealing the opening by the first end of the pipe body 15. The side wall 1504 of the pipe body 15 can fold or unfold axially to achieve volume change within the cylinder body 12.

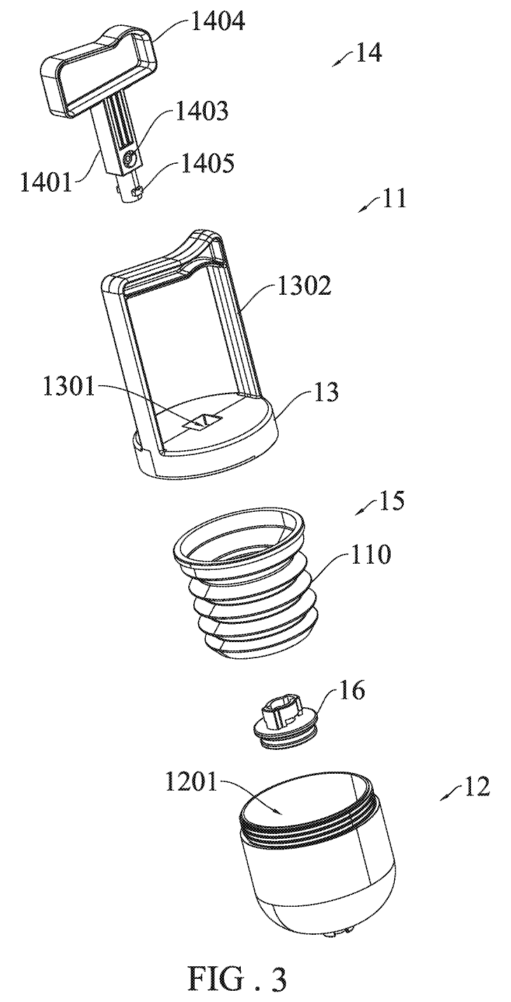

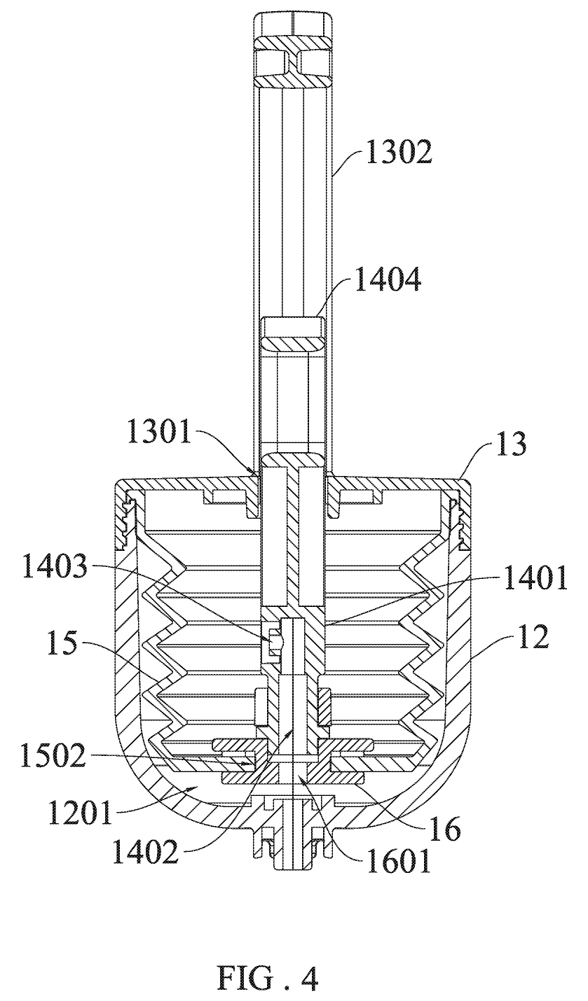

In some embodiments, as shown in FIGS. 3 and 4, the variable-volume structure 11 further includes a pull rod assembly 14 including a rod body 1401. A first end of the rod body 1401 is inserted through the first end of the pipe body 15 and connected to the second end of the pipe body 15. The rod body 1401 is movable reciprocally along the axial direction of the pipe body 15, causing the second end of the pipe body 15 to move accordingly and fold or unfold the side wall 1504.

In some embodiments, as shown in FIG. 4, the first end of the cylinder body 12 is arranged with a cover 13. An inner diameter of the cover 13 is greater than the outer diameter of the flange 1503 of the pipe body 15. When the cover 13 is fastened to the first end of the cylinder body 15, the cover 13 further compresses the flange 1503 against the opening of the cylinder body 12, enhancing sealing effect. A top of the cover 13 defines a first through hole 1301 for the rod body 1401 to pass through.

In some embodiments, as shown in FIG. 4, an inner wall of the cover 13 has internal threads, and the first end of the cylinder body 12 has external threads, allowing threaded connection. Alternatively, the cover 13 and the first end of the cylinder body 12 may be connected by snapping or adhesion.

In some embodiments, as shown in FIG. 4, an inner wall of the first through hole 1301 and an outer wall of the rod body 1401 have a gap fit, restricting the movement direction of the rod body 1401.

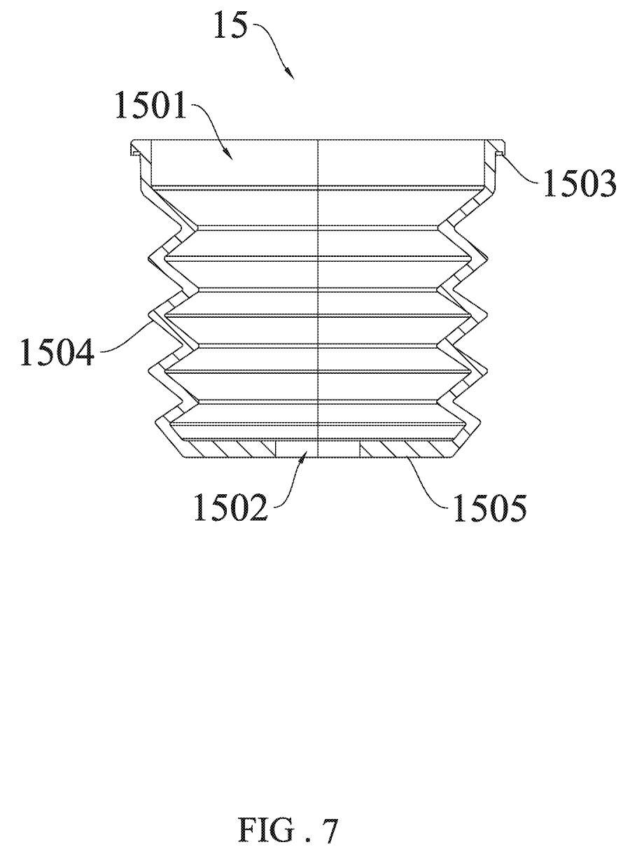

In some embodiments, as shown in FIG. 7, the bottom wall 1505 of the pipe body 15 defines a second through hole 1502 to be connected to a connecting member 16. The connecting member 16 is a columnar body with an inwardly recessed ring groove 1602 on a side wall. The second through hole 1502 of the pipe body 15 engages with the ring groove 1602, fixing the connecting member 16 to the bottom wall 1505. The first end of the rod body 1401 is connected to the connecting member 16.

As shown in FIG. 1, the liquid storage tank 20 requires sufficient suction from the air extraction device 10. Thus, the air extraction device 10 is required to draw in enough air through the first air hole 1202 to provide adequate suction for the liquid storage tank 20, which is connected to the first air hole 1202, to draw nasal mucus. However, it is unnecessary to expel air through the first air hole 1202. To this end, in some embodiments, a second air hole 1601 is defined on the bottom wall 1505 of the pipe body 15, connecting the cavity 1201 of the cylinder body 12 to the inner cavity 1501 of the pipe body 15. The second air hole 1601 is connected to a first one-way valve 111, allowing unidirectional air expulsion from the cavity 1201. Alternatively, the second air hole 1601 may be disposed on the cylinder body 12, connecting the cavity 1201 to the exterior of the cylinder body 12. The second air hole 1601 is connected to a first one-way valve 111, allowing unidirectional expulsion air expulsion from the cavity 1201.

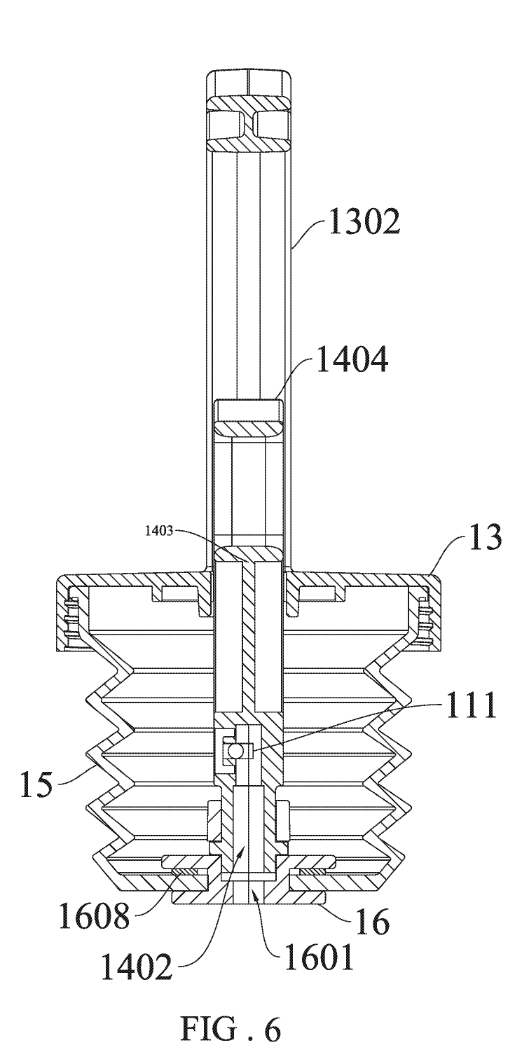

As shown in FIG. 6, in some embodiments, the second air hole 1601 is disposed on the connecting member 16. The first end of the rod body 1401 is arranged with an air passage 1402, and a side wall of the rod body 1401 defines a third air hole 1403. A first end of the air passage 1402 is in communication with the second air hole 1601, and a second end of the air passage 1402 is in communication with the third air hole 1403, with the first one-way valve 111 arranged on the third air hole 1403.

During the expansion of the pipe body 15, the air pressure in the cavity 1201 rises, expelling part of the air within the cavity 1201 through the second air hole 1601. However, some air may still exit through the first air hole 1202. Thus, in some embodiments, the first air hole 1202 is connected to a second one-way valve 2104, allowing unidirectional air intake into the cavity 1201. Under the control of the first one-way valve 111 and second one-way valve 2104, the air extraction device 10 may provide suction only to the liquid storage tank 20.

For ease of use, as shown in FIGS. 2 and 3, in some embodiments, the top of the cover 13 is arranged with a first handle 1302 designed as a ring structure. The second end of the rod body 1401 is arranged with a second handle 1404, which is movable reciprocally within a ring defined by the first handle 1302. Users can hold both the first handle 1302 and the second handle 1404 with one hand and pull the rod to perform a suction action.

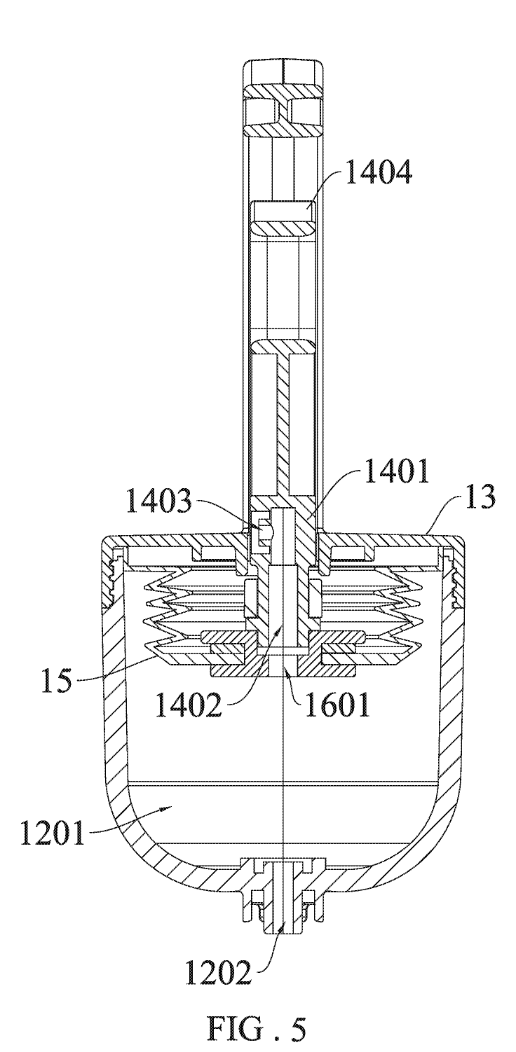

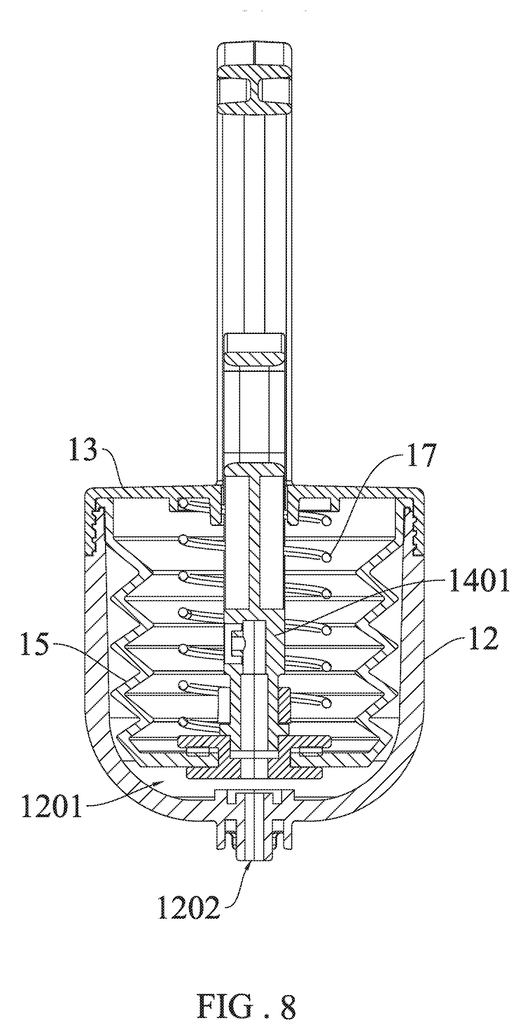

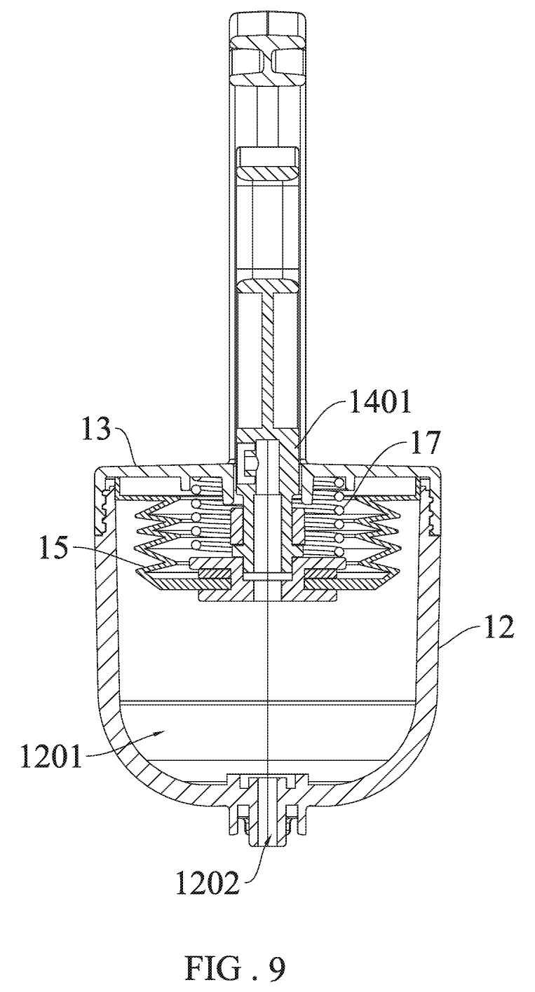

As shown in FIGS. 4 and 5, in the above embodiments, the expansion and contraction of the pipe body 15 are manually operated. In some embodiments, as shown in FIGS. 8 and 9, a spring 17 is arranged in the inner cavity 1501 of the pipe body 15. A first end of the spring 17 abuts against the cover 13, and a second end of the spring 17 abuts against the bottom wall 1505 of the pipe body 15. The spring may have three states: (1) a normal state without external force; (2) a compressed state under external force; and (3) an extended state under external force. For example, assuming that the spring in FIG. 8 is in the normal state, pulling the rod body 1401 upward (FIG. 9) will compress the spring 17 and contracts the pipe body 15. Releasing the force allows the spring 17 to return to normal, expanding the pipe body 15 (FIG. 8). This simplifies operation by requiring only an upward pull.

Secondly, in some embodiments, assuming that the spring 17 in FIG. 9 is in the normal state. As shown in FIG. 8, a downward force is applied to push the rod body1401, causing the pipe body 15 to extend and the spring 17 to transition to the extended state. Upon removal of the downward force, as depicted in FIG. 9, the spring 17 actively returns to the normal state, contracting the pipe body 15.

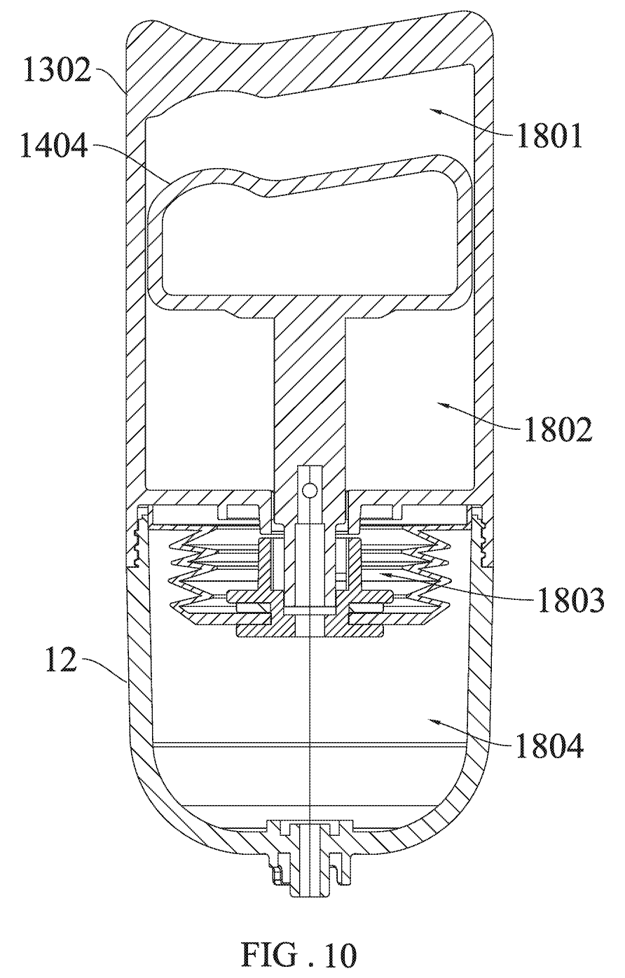

As shown in FIG. 10, the air extraction device 10 may include four installation areas: a first area 1801 between the first handle 1302 and the second handle 1404, a second area 1802 between the cover 13 and the second handle 1404, a third area 1803 between the bottom wall 1505 of the pipe body 15 and the cover 13, and a fourth area 1804 between the bottom of the cylinder body 12 and the bottom wall 1505 of the pipe body 15. In the above embodiments, the spring 17 is arranged within the third area 1803. Alternatively, the spring 17 may be arranged within other installation areas to similarly achieve the function performed by the spring 17 in the aforementioned embodiments.

Furthermore, the spring 17 in the above embodiments may be replaced by other elastic components possessing restorative capability, such as an elastic strap.

In some embodiments, the pipe body 15 has self-recovery capabilities. The structure of the pipe body 15 includes but is not limited to the following examples: Example 1: the side wall 1504 of the pipe body 15 features a foldable structure as shown in FIG. 4. The normal state of this foldable structure is either an extended state or a folded state. Under external force, the pipe body 15 can transform into either a folded or extended state, and upon removal of the external force, it can automatically revert to its extended or folded state. Example 2: The side wall 1504 of the pipe body 15 is made of an elastic material. The pipe body 15 can be stretched under external force and automatically returns to its normal state upon removal of the external force.

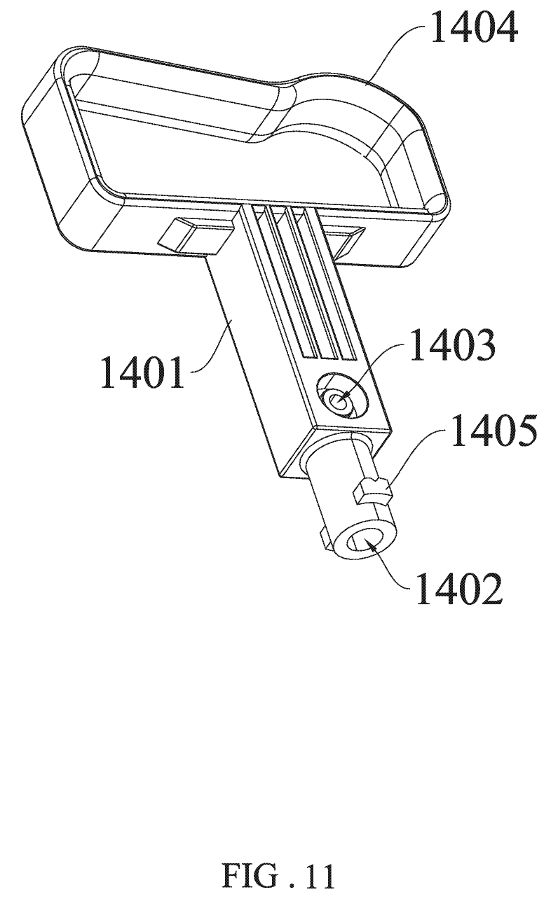

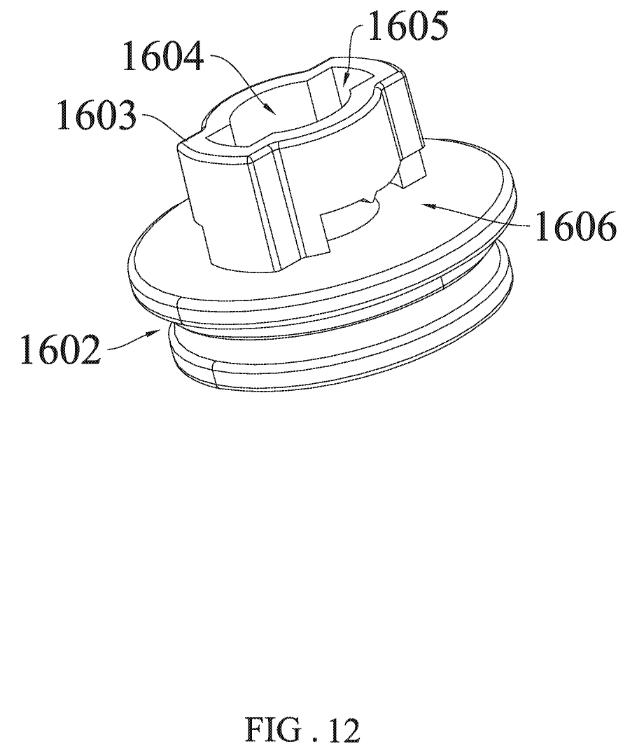

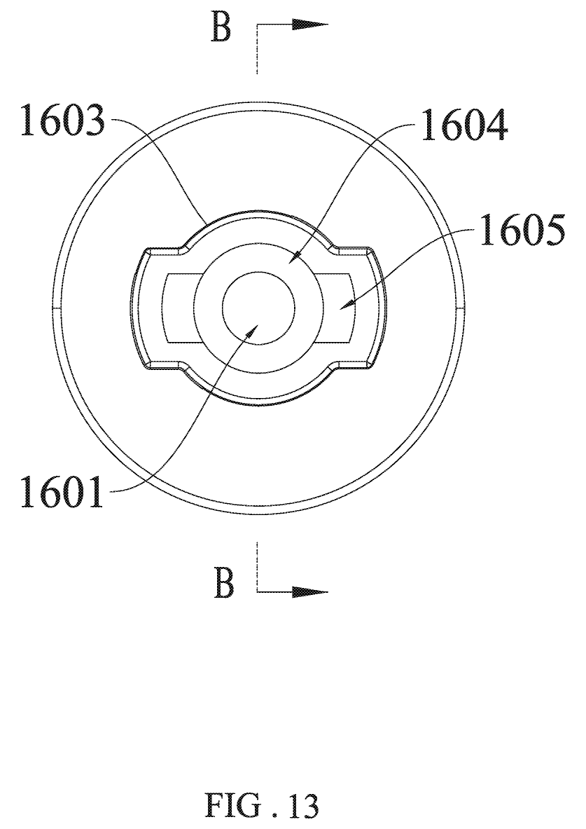

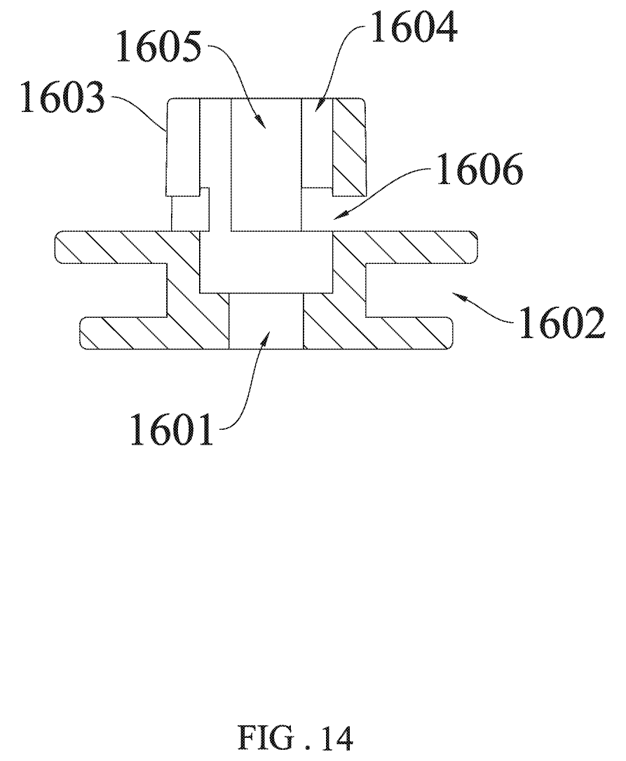

As shown in FIGS. 11 to 14, in some embodiments, a detachable connection structure is provided. The connection structure includes a locking block 1405 disposed on the side wall of the first end of the rod body 1401. The connection structure further includes a connecting column 1603 disposed on the connecting member 16. The connecting column 1603 defines an insertion hole 1604, and the insertion hole 1604 is in communication with the second air hole 1601. In the embodiments, the insertion hole 1604 and the second air hole 1601 share the same axial direction. A side wall of the insertion hole 1604 defines a guide groove 1605. A side wall of the connecting column 1603 on an end near the connecting member 16 defines a lock hole 1606, and the lock hole 1606 is opened along a peripheral direction of the connecting column 1603. An end of the guide groove 1605 near the connecting member 16 is in communication with a first end of the lock hole 1606. When connecting the rod body 1401 and the connecting member 16, the first end of the rod body 1401 may be inserted into the insertion hole 1604, and the locking block 1405 on the rod body 1401 can move along the guide groove 1605 to a set position. By rotating the rod body 1401 or the connecting member 16, the locking block 1405 moves towards a second end of the lock hole 1606. This completes the connection between the rod body 1401 and the connecting member 16, and they are locked relative to each other in the axial direction. The disassembly of the rod body 1401 and the connecting member 16 is performed by reversing the above process. When the end of the guide groove 1605 near the connecting member 16 is in communication with the middle of the lock hole 1606, after the locking block 1405 on the rod body 1401 moves along the guide groove 1605 to the set position, the rod body 1401 or the connecting member 16 can be rotated in either of two directions, causing the locking block 1405 to move towards either the first end or the second end of the lock hole 1606.

Embodiments of the present disclosure disclose a liquid storage tank 20, which is configured to extract nasal mucus. A first end of the liquid storage tank 20 defines a liquid inlet hole 2101. The liquid storage tank 20 is arranged with a chamber 23 for storing nasal mucus, and the liquid inlet hole 2101 is in communication with the chamber 23. An outer wall of the liquid storage tank 20 defines a fourth air hole 2103, and the fourth air hole 2103 is in communication with the chamber 23. A first end of the air guide pipe 30 is connected to the first air hole 1202 of the air extraction device 10, and a second end of the air guide pipe 30 is connected to the fourth air hole 2103 of the liquid storage tank 20. During use, the first end of the liquid storage tank 20 is placed near or inserted into the nostril. When the air extraction device 10 provides sufficient suction to the liquid storage tank 20, nasal mucus will be extracted and flow into the chamber 23 through the liquid inlet hole 2101.

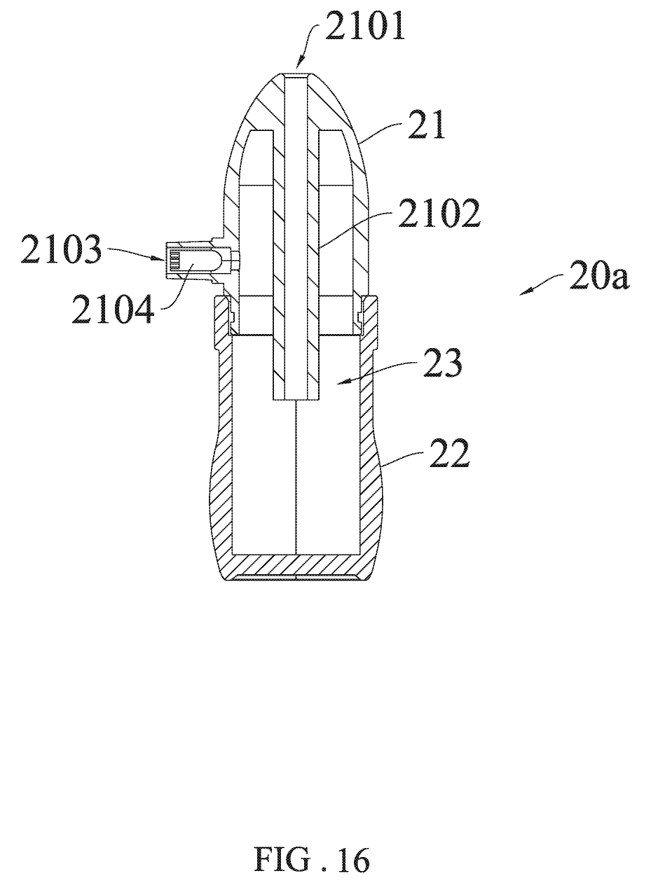

In some embodiments, as shown in FIG. 16, the second one-way valve 2104 described in the above embodiments may be selectively disposed on the fourth air hole 2103.

In some embodiments, as shown in FIG. 16, a conduit 2102 is disposed within the chamber 23 of the liquid storage tank 20. A first end of the conduit 2102 is connected to the liquid inlet hole 2101. Compared to the fourth air hole 2103, a second end of the conduit 2102 is closer to a bottom of the chamber 23. Its purpose is to guide the nasal mucus, after entering through the liquid inlet hole 2101, into the chamber 23 via the conduit 2102, thereby limiting or preventing the nasal mucus from entering the fourth air hole 2103 during its flow into the chamber 23, which could cause blockage of the air guide pipe 30 or contamination of the air guide pipe 30 and the air extraction device 10.

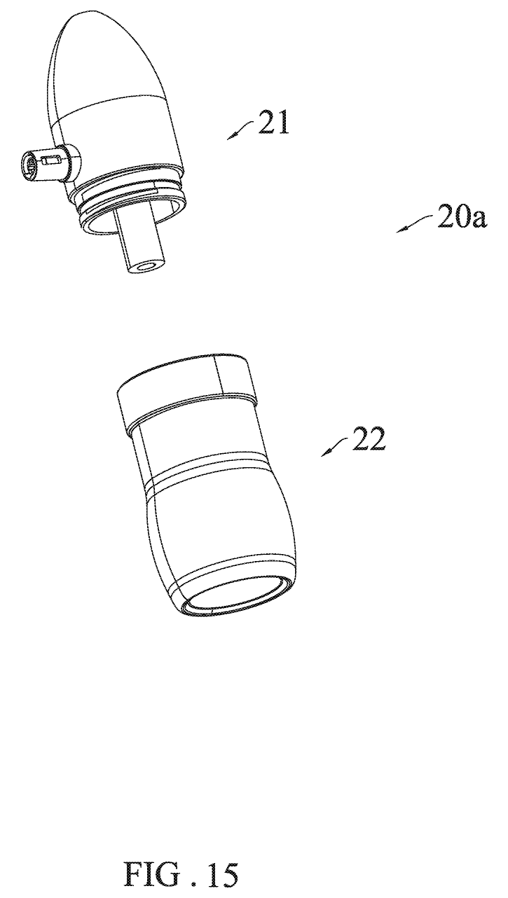

In some embodiments, as shown in FIGS. 15 and 16, the liquid storage tank 20a has a split structure. The liquid storage tank 20a includes a head 21 and a tank body 22, which are detachably connected. The split structure makes cleaning the liquid storage tank 20a relatively easier and allows for more thorough cleaning.

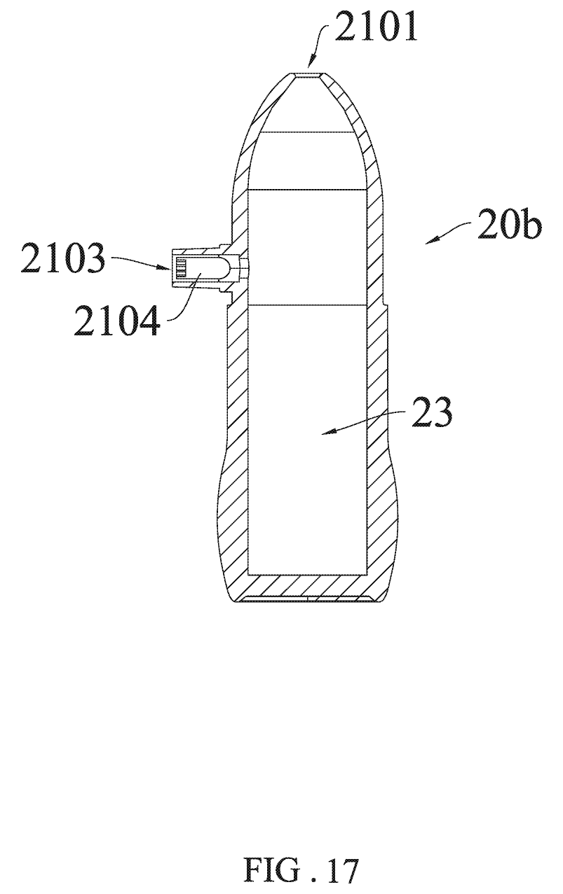

Furthermore, in some embodiments, as shown in FIG. 17, the liquid storage tank 20b may be an integrated structure. It can be selectively used as a disposable application to prevent cross-contamination. Alternatively, if the integrated liquid storage tank 20b is not cleaned thoroughly, some viruses may remain on the liquid storage tank 20, leading to repeated infections. Of course, the integrated liquid storage tank 20b may be reused.

Compared with the related art, the embodiments of the present disclosure have the following advantages: the air extraction device includes a cylinder body and a variable-volume body. The cylinder body is arranged with a cavity and a first air hole, and the first air hole is in communication with the cavity. The variable-volume body is disposed within the cavity, and its volume can selectively change within the cavity. The air pressure in the cavity is changed through the volume change of the variable-volume body, and by the transformation of the variable-volume body's volume from large to small, the cavity provides suction force externally through the first air hole. During the process of providing suction force, the outer wall of the variable-volume body may not contact the inner wall of the cylinder body, thereby avoiding friction between them and improving the service life of the device. Additionally, in some embodiments, an elastic member may be set to assist the air extraction operation. The user only needs to provide pulling or pushing force, making the operation simpler and more convenient. Third, in some embodiments, the first air hole is connected to a second one-way valve that only allows gas to enter the cavity unidirectionally; the second air hole is connected to a first one-way valve that only allows gas to be discharged unidirectionally from the cavity. The cooperation of the two one-way valves controls the airflow direction of the air extraction device, enabling the air extraction device to provide suction force only for the liquid storage tank, making it easier for the nasal aspirator to extract nasal mucus. Fourth, the rod body and the connecting piece in the embodiment are detachably connected, which may improve the assembly efficiency of the air extraction device. Fifth, the liquid storage tank may be a split structure or an integrated structure. When cleaning the liquid storage tank, the split structure is relatively easier to clean more thoroughly, which is beneficial for repeated use. However, the integrated structure can be selectively used as a disposable application to prevent cross-contamination; or if the liquid storage tank is not cleaned thoroughly, some viruses may remain on the liquid storage tank, leading to repeated infections.

The above descriptions are merely some embodiments of the present disclosure and are not intended to limit the present disclosure. For those skilled in the art, various modifications and changes may be made to the present disclosure. Any modifications, equivalent substitutions, improvements, etc., made within the spirit and principles of the present disclosure shall be included within the scope of the present disclosure.

What is claimed is:

1. An air extraction device, comprising:
a cylinder body; wherein the cylinder body defines a cavity and a first air hole, and the first air hole is in communication with the cavity; and
a variable-volume body, arranged within the cavity;
wherein the variable-volume body is configured such that, through a change in a volume of the variable-volume body within the cavity from large to small, an air pressure within the cavity is reduced, for providing a suction force externally through the first air hole.

2. The air extraction device according to claim 1, wherein the cylinder body defines an opening;
the variable-volume body comprises a pipe body; wherein the pipe body comprises a first end and a second end, and the second end of the pipe body is arranged with a bottom wall;
wherein the first end of the pipe body is capable of sealing the opening of the cylinder body, and a side wall of the pipe body is capable of expanding and of contracting within the cavity.

3. The air extraction device according to claim 2, wherein the first end of the pipe body is arranged with a flange that is annular in shape and extends radially outward; an outer diameter of the flange is greater than an inner diameter of the opening of the cylinder body, and the first end of the pipe body is capable of sealing the opening of the cylinder body via the flange; and/or
the side wall of the pipe body is capable of expanding and of folding along an axial direction of the pipe body.

4. The air extraction device according to claim 2, wherein the air extraction device further comprises a pull rod assembly; the pull rod assembly comprises a rod body, and a first end of the rod body is inserted through the first end of the pipe body and connected to the bottom wall of the pipe body;
the rod body is configured to drive the pipe body to expand or contract by pushing or pulling the rod body.

5. The air extraction device according to claim 3, wherein the opening of the cylinder body is arranged with a cover, and the cover is configured to restrict the flange at the opening of the cylinder body.

6. The air extraction device according to claim 5, wherein a top of the cover is arranged with a first handle designed as a ring structure; a second end of the rod body is arranged with a second handle that is movable reciprocally within a ring defined by the first handle.

7. The air extraction device according to claim 5, wherein a spring is arranged within the pipe body; a first end of the spring abuts against the cover, and a second end of the spring abuts against the bottom wall of the pipe body.

8. The air extraction device according to claim 5, wherein a top of the cover defines a first through hole for the rod body to pass through, and an inner wall of the first through hole and an outer wall of the rod body have a gap fit.

9. The air extraction device according to claim 4, wherein the air extraction device further comprises a second air hole and a first one-way valve;
the second air hole is disposed on the pipe body or the cylinder body; a first end of the second air hole is in communication with the cavity of the cylinder body, and a second end of the second air hole is connected to the first one-way valve; wherein gas within the cavity is capable of being discharged unidirectionally through the first one-way valve.

10. The air extraction device according to claim 9, wherein the bottom wall of the pipe body defines a second through hole; the second through hole is arranged with a connecting member, and the second air hole is disposed on the connecting member;
the rod body is arranged with an air passage, and a side wall of the rod body defines a third air hole; wherein a first end of the air passage is in communication with the second air hole, and a second end of the air passage is in communication with the third air hole.

11. The air extraction device according to claim 10, wherein an outer wall of the first end of the rod body is arranged with a locking block;
the connecting member is arranged with a connecting column, and the connecting column defines an insertion hole; wherein the insertion hole is in communication with the second air hole; a side wall of the insertion hole defines a guide groove along an axial direction of the insertion hole; a side wall of the connecting column near the connecting member defines a lock hole, and the lock hole is opened along a peripheral direction of the connecting column; wherein the guide groove is in communication with the lock hole;
the rod body is capable of being inserted into the insertion hole, and the locking block is movable along the guide groove to a set position; in a case where the rod body is relatively rotated, the locking block moves toward an end of the lock hole.

12. The air extraction device according to claim 7, wherein the air extraction device further comprises an elastic member; the elastic member is configured to drive the variable-volume body to expand or contract through a restoring force of the elastic member.

13. The air extraction device according to claim 1, wherein the first air hole is connected to a second one-way valve, and the cavity of the cylinder body is capable of unidirectionally drawing in air through the second one-way valve.

14. A nasal aspirator, comprising:
a liquid storage tank, an air guide pipe, and an air extraction device;
wherein the air extraction device comprises:
a cylinder body; wherein the cylinder body defines a cavity and a first air hole, and the first air hole is in communication with the cavity; and
a variable-volume body, arranged within the cavity;
wherein the variable-volume body is configured such that, through a change in a volume of the variable-volume body within the cavity from large to small, an air pressure within the cavity is reduced, for providing a suction force externally through the first air hole;
wherein the liquid storage tank comprises a chamber; a first end of the liquid storage tank defines a liquid inlet hole, and the liquid inlet hole is in communication with the chamber; a side wall of the liquid storage tank defines a fourth air hole, and the fourth air hole is in communication with the chamber;
a first end of the air guide pipe is in communication with the first air hole of the air extraction device, and a second end of the air guide pipe is in communication with the fourth air hole of the liquid storage tank.

15. The nasal aspirator according to claim 14, wherein the liquid storage tank further comprises a conduit; the conduit is disposed within the chamber, and a first end of the conduit is connected to the liquid inlet hole.

16. The nasal aspirator according to claim 14, wherein the cylinder body defines an opening;
the variable-volume body comprises a pipe body; wherein the pipe body comprises a first end and a second end, and the second end of the pipe body is arranged with a bottom wall;
wherein the first end of the pipe body is capable of sealing the opening of the cylinder body, and a side wall of the pipe body is capable of expanding and of contracting within the cavity.

17. The nasal aspirator according to claim 16, wherein the first end of the pipe body is arranged with a flange that is annular in shape and extends radially outward; an outer diameter of the flange is greater than an inner diameter of the opening of the cylinder body, and the first end of the pipe body is capable of sealing the opening of the cylinder body via the flange; and/or
the side wall of the pipe body is capable of expanding and of folding along an axial direction of the pipe body.

18. The nasal aspirator according to claim 16, wherein the air extraction device further comprises a pull rod assembly; the pull rod assembly comprises a rod body, and a first end of the rod body is inserted through the first end of the pipe body and connected to the bottom wall of the pipe body;
the rod body is configured to drive the pipe body to expand or contract by pushing or pulling the rod body.

19. The nasal aspirator according to claim 17, wherein the opening of the cylinder body is arranged with a cover, and the cover is configured to restrict the flange at the opening of the cylinder body.

20. The nasal aspirator according to claim 18, wherein the air extraction device further comprises a second air hole and a first one-way valve;
the second air hole is disposed on the pipe body or the cylinder body; a first end of the second air hole is in communication with the cavity of the cylinder body, and a second end of the second air hole is connected to the first one-way valve; wherein gas within the cavity is capable of being discharged unidirectionally through the first one-way valve.
